(19) United States
(12) Patent Application Publication
(10) Pub. No.: US 2025/0147037 A1
(43) Pub. Date: May 8, 2025

(54) KIT FOR ANALYZING INTERACTOMES, AND METHOD FOR ANALYZING INTERACTOMES BY USING SAME

(71) Applicants: O2MEDI INC., Ulsan (KR); UNIST (ULSAN NATIONAL INSTITUTE OF SCIENCE AND TECHNOLOGY), Ulsan (KR)

(72) Inventors: Tae Hyuk KWON, Ulsan (KR); Jung Seung NAM

(21) Appl. No.: 18/832,634

(22) PCT Filed: Dec. 6, 2022
(86) PCT No.: PCT/KR2022/019687

§ 371 Date: Jul. 24, 2024

(30) Foreign Application Priority Data
Jan. 28, 2022 (KR) 10-2022-0013509

(51) Int. Cl. G01N 33/68; B01J 23/46; C12N 9/14; C12N 15/85; G01N 33/58

(52) U.S. Cl. CPC G01N 33/6845; B01J 23/468; C12N 9/14; C12N 15/85; G01N 33/582; G01N 33/6848; C07K 2319/09; C12Y 308/01005

(57) ABSTRACT

The present invention relates to a kit for analyzing interactomes, and a method for analyzing interactomes through photo-crosslinking by using the kit. According to the kit for analyzing interactomes and the method for analyzing interactomes by using same, in one aspect of the present invention, interactomes are linked through a covalent bond one by one so that analysis errors can be reduced. In addition, according to the present invention, interactomes in living cells can be analyzed and interactomes in the nucleus of cells can be analyzed. Furthermore, according to the present invention, the formation time point of interactomes can be regulated so that target proteins and interactomes can be analyzed in a specific situation or time point.

### TECHNICAL FIELD

The present invention relates to a kit for interactomes and method for analyzing interactomes through photo-crosslinking by using the kit.

### BACKGROUND ART

In order to understand and treat human diseases, an accurate understanding of the physiological functions of cells, the basic units that make up humans, is necessary. In addition, since most physiological functions of cells are regulated by protein-protein interaction following protein expression, it is imperative to understand which proteins interact with each other and how these interactions are involved. The disciplines that understand these proteins and identify interactions between proteins are called proteomics and interactomics, and research in these disciplines has been actively underway recently.

There are known methods for proteomics and interactomics research, including (i) immunoprecipitation, (ii) enzyme-based labeling, and (iii) chemical reaction-based labeling (see FIGS. 1A to 1E).

The interactome analysis methods reported to date have limitations in that they can only be applied to specific proteins of interest (POI), cannot be used in living cells, and have very low selectivity, sensitivity, and spatiotemporal resolution for the interactome (see FIG. 1F).

The first proposed methodology for the isolation and analysis of interactomes is immunoprecipitation using antibodies that can specifically attach to POI. However, because the immunoprecipitation method uses physical adsorption between POI and interacting proteins, not only the interacting proteins but also other surrounding proteins are adsorbed together, resulting in a high possibility of false-positive. Thus, there was a limitation in that the spatiotemporal resolution and selectivity for interactome analysis were very low (see FIG. 1A).

Accordingly, a method developed to overcome the limitations of immunoprecipitation is chemical labeling, which utilizes chemical modification that occurs locally between proteins and chemicals. Representative methods for causing chemical modification of proteins include enzyme-based labeling and chemical reaction-based labeling (FIGS. 1B to 1E).

In the case of enzyme-based labeling, biotin-phenoxyl radical, an intermediate product usually generated by electron transfer between biotin-phenol and enzyme, is attached to surrounding proteins, so it can effectively label interactants. However, due to the long lifetime and wide diffusion distance of the biotin-phenoxyl radical, there was limitation in that the spatiotemporal resolution and selectivity for interactomes were low, with a labeling radius of about 70 to 100 nm (see FIG. 1B).

A method proposed as an alternative to this is a chemical reaction-based labeling method, and representative chemical reaction-based labeling methods include (i) μMap method, (ii) ligand direct chemical method, and (iii) chemical crosslinking method (see FIGS. 1C to 1E).

In the case of the μMap method, the labeling radius was greatly improved from tens of nm to several nm compared to the enzyme-based labeling method by using a carbene intermediate with a short survival life. However, since the target protein is targeted using an antibody, it is difficult to apply to the intracellular region, so the applicable target protein range is very limited (FIG. 1C).

In the case of the direct ligand chemical method, POI labeling is possible using only chemical reactions without the intracellular expression of additional proteins. However, customized chemicals are required for each POI, making analysis impossible for POIs for which ligands have not been developed (FIG. 1D).

In the case of the chemical crosslinking method, it can be applied to identified cell lysates, but it has limitations in that because indiscriminate chemical reactions cause overall crosslinking of proteins other than POI, there is no spatiotemporal resolution and it is difficult to apply to living cells (FIG. 1E).

Therefore, the present inventors and research team would like to present a new proteomics interactomics analysis method with high precision, high sensitivity, and high resolution that uses photo-crosslinking (FIG. 2) as a protein chemical modification method that can overcome the disadvantages of the existing technologies above and can be applied to POIs located in the nucleus, which are chemically difficult to access in living cells.

### DISCLOSURE

### Technical Problem

In one aspect, an object of the present invention is to improve the shortcomings of existing interactome analysis methods.

In one aspect, an object of the present invention is to precisely analyze protein-protein interactions.

In one aspect, an object of the present invention is to improve accuracy in interactome analysis.

In one aspect, an object of the present invention is to provide a method for analyzing interactomes without temporal or spatial constraints.

In one aspect, an object of the present invention is to perform interactome analysis in cells.

In one aspect, an object of the present invention is to analyze interactomes present in living cells.

In one aspect, an object of the present invention is to analyze interactomes located in the nucleus of living cells.

### Technical Solution

In order to achieve the above objects, one aspect of the present invention provides a kit for analyzing an interactome, comprising i) a first vector comprising a protein of interest (POI) coding portion and a fluorescent protein coding portion; ii) a second vector comprising a fluorescent protein binding protein coding portion and a HaloTag coding portion; and iii) a photocatalyst comprising a HaloTag targeting ligand (HTL).

In addition, one aspect of the present invention provides a method for analyzing an interactome using a photo-crosslinking using the kit for analyzing the interactome, the method comprising the steps of i) expressing in a cell a first construct comprising a protein of interest (POI) and a fluorescent protein, and a second construct comprising a fluorescent protein binding protein, a HaloTag, and a nuclear localization sequence, by using the first vector and the second vector; ii) treating the cell with a photocatalyst comprising a HaloTag targeting ligand; iii) irradiating a light source to induce a photo-crosslinking chemical reaction between the POI and the interactome; and iv) extracting photo-crosslinked proteins.

### Advantageous Effects

In the case of the kit for analyzing an interactome and method for analyzing an interactome using the same according to one aspect of the present invention, analysis errors can be reduced because the interactomes are linked together through covalent bonds. In addition, according to the present invention, interactomes within living cells can be analyzed, and interactomes within the cell nucleus can be analyzed. In addition, according to the present invention, the point of interactome formation can be controlled, so there is an advantage in that it is possible to analyze the protein of interest and the interactome in a specific situation or time point.

### DESCRIPTION OF DRAWINGS

FIG. 1A is a diagram showing a mechanism of a conventional immunoprecipitation method.

FIG. 1B is a diagram showing a mechanism of a conventional enzyme-based labeling method.

FIG. 1C is a diagram showing a mechanism of a Map method among conventional chemical reaction-based labeling methods.

FIG. 1D is a diagram showing a mechanism of a ligand direct chemical method among conventional chemical reaction-based labeling methods.

FIG. 1E is a diagram showing a mechanism of a chemical crosslinking method among conventional chemical reaction-based labeling methods.

FIG. 1F shows a comparison of a difference in effectiveness between the present invention and conventional techniques.

FIG. 2 is an example of a mechanism of photo-crosslinking according to the present invention.

FIG. 3 is a diagram explaining an effect of combined use of a HaloTag system and a GFP-GBP system according to the present invention.

FIG. 4 is a diagram showing a process of an interactome analysis method using photo-crosslinking, which is an embodiment of the present invention.

FIG. 5 is a photograph showing a result of detection of RAVER1, MART3, and SFPQ, which are interactomes of PTBP1, according to an analysis method of the present invention.

FIG. 6 is a photograph showing a result of confirming an interaction of PTBP1-RAVER1.

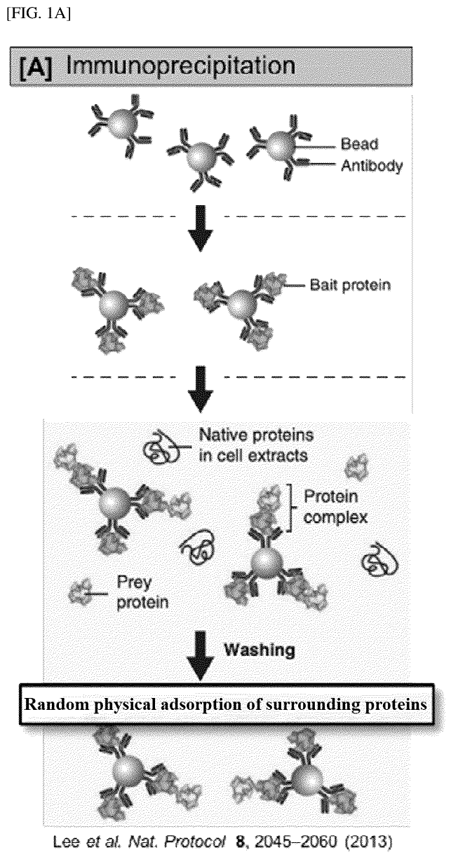

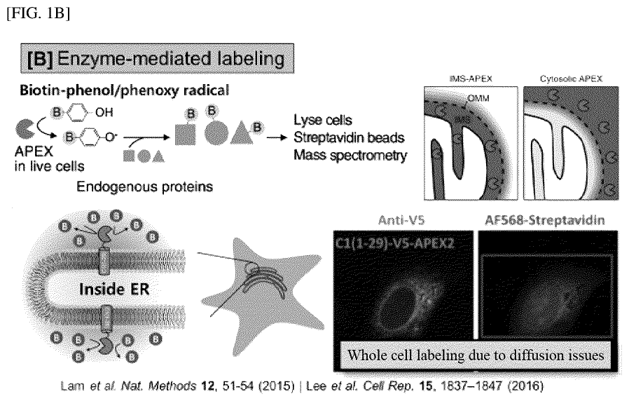

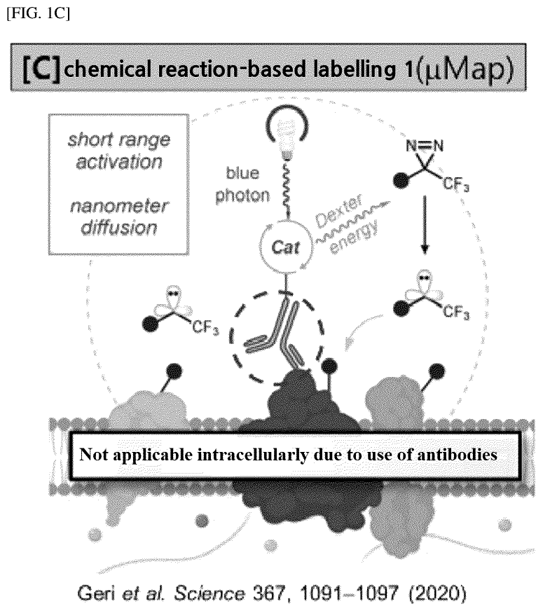

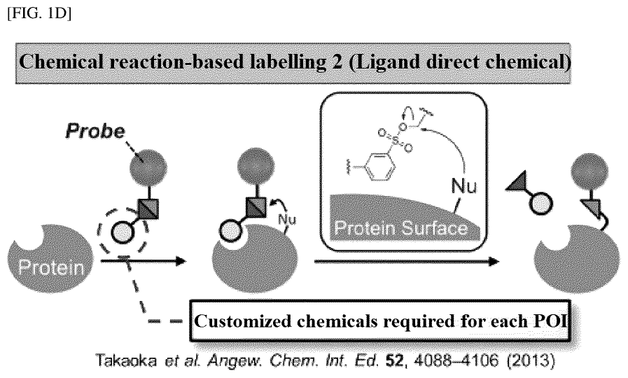

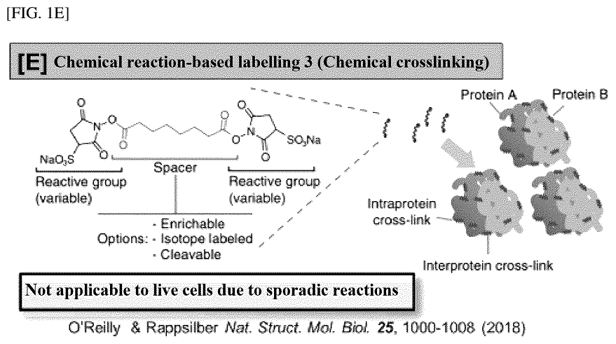

### MODE FOR INVENTION

Hereinafter, each configuration will be described in more detail.

The following description is only an example, and the scope of the present invention is not limited by the following.

In addition, it is to be understood that the terms or words used in the specification and claims of the present invention are not to be construed in a conventional or dictionary sense, and that the inventors can properly define the concept of a term to describe their invention in the best possible way. Accordingly, the present invention should be construed as having a meaning and concept consistent with the technical idea of the present invention.

In the specification of the present invention, when a portion “includes” any component, this means that the portion does not exclude other components, but may further include other components unless otherwise stated.

According to one aspect, the present invention is a kit for analyzing an interactome, comprising i) a first vector comprising a protein of interest (POI) coding portion and a fluorescent protein coding portion; ii) a second vector comprising a fluorescent protein binding protein coding portion and a HaloTag coding portion; and iii) a photocatalyst comprising a HaloTag targeting ligand (HTL).

The first and second vectors are not limited and may include plasmids, bacteriophages, cosmids, bacterial artificial chromosomes (BACs), yeast artificial chromosomes (YACs), viruses, etc.

The vector may include factors that the vector must have for protein expression, such as origin of replication, multiple cloning sites, selection marker, etc.

In one aspect, the kit may be in a form comprising proteins formed by expressing the vectors, and specifically, the kit may comprise a conjugate of a target protein and a fluorescent protein; a conjugate of a fluorescent protein binding protein, a HaloTag, and a nuclear location sequence; and a photocatalyst comprising a HaloTag targeting ligand.

In one aspect, the fluorescent protein may include a green fluorescent protein (GFP), and the fluorescent protein binding protein may include a green fluorescent protein binding protein (GBP), but there are not limited thereto.

In one aspect, the present invention can analyze POIs and interactomes for each detailed location by using both the HaloTag system and the GFP-GBP system (see FIG. 3). In other words, by including the HaloTag system, the photocatalyst combined with the HaloTag target ligand can bind specifically to the HaloTag protein. In case where only the HaloTag is used, the GFP-GBP system has been also introduced to compensate for the shortcoming of not being able to remove background signals from other proteins detected together with the interactome during protein analysis.

Additionally, the photocatalyst may include a conjugate of a metal and a Halotag ligand (HTL).

Additionally, in one aspect, the metal may include iridium.

The present invention can perform photo-crosslinking within cells by using a catalyst including iridium.

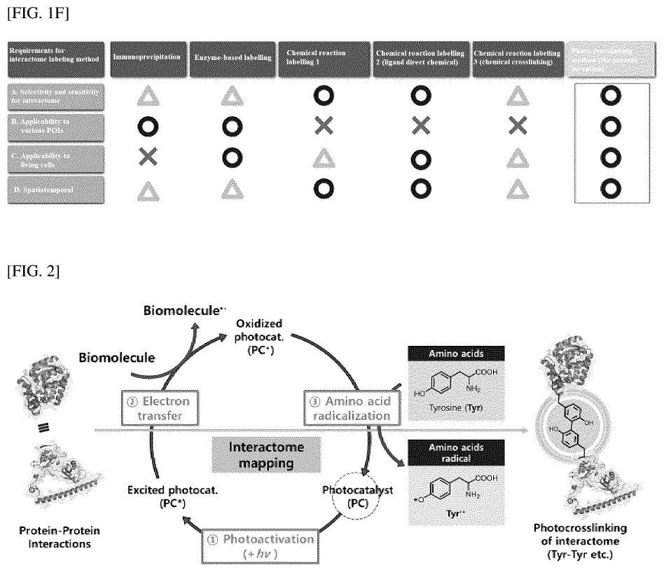

Meanwhile, by utilizing the photo-crosslinking, the present invention can freely control the timing of labeling the interactome using light (see FIG. 2).

In one aspect, the kit may be for analyzing interactomes within cells or may be for analyzing interactomes present in the nucleus.

In one aspect, the POI may be PTBP1, POU2F1, or PSMA2, but is not limited thereto.

In one aspect, the second vector may further include a nuclear localization sequence (NLS) coating portion.

In one aspect, the present invention is an interactome analysis method using the photo-crosslinking using the above interactome analysis kit.

An example of the mechanism of the photo-crosslinking according to the present invention is shown in FIG. 2.

In particular, a method for analyzing an interactome according to the present invention may comprise the steps of i) expressing in a cell a first construct comprising a protein of interest (POI) and a fluorescent protein, and a second construct comprising a fluorescent protein binding protein, a HaloTag, and a nuclear localization sequence, by using the first vector and the second vector; ii) treating the cell with a photocatalyst comprising a HaloTag targeting ligand; iii) irradiating a light source to induce a photo-crosslinking chemical reaction between the POI and the interactome; and iv) extracting photo-crosslinked proteins.

In one aspect, the first structure may refer to a product (or protein complex) of the first vector expressed, and the second structure may refer to a product of the second vector expressed.

In one aspect, the expressed second structure may bind to a photocatalyst including a HaloTag targeting ligand, and then the complex may bind to the expressed first structure again.

In one aspect, by irradiating a light source, the photocatalyst including a metal, such as iridium, may be photoactivated, thereby causing a photo-crosslinking chemical reaction between the POI and the interactome.

The assay may further include the step of lysing the cells after the photo-crosslinking chemical reaction occurs between the POI and the interactome, as described above.

In one aspect, the analysis method may further comprise the step of extracting photo-crosslinked proteins after lysing the cells.

The extraction step may use biotinylation. Specifically, it may include a method of biotinylating the HaloTag protein, for example, the method described in Korean Patent Publication No. 10-2019-0017363 can be used.

Thereafter, the analysis method may further include the step of enriching the biotinylated protein from the cell lysate using streptavidin (SA) magnetic beads.

In one aspect, the analysis method may further include, after separating the biotinylated protein, the step of removing non-specific proteins adsorbed to the biotinylated protein due to ionic bonds, hydrophobic bonds, etc. Here, the step of removing the non-specific proteins may include SDS-denaturation.

In one aspect, the analysis method may further include the step of analyzing the protein including the interactome through mass spectrometry after removing the non-specifically adsorbed protein.

Here, the method may further comprise the step of treating with trypsin prior to mass spectrometry to decompose the biotinylated protein into constituent peptides or proteins.

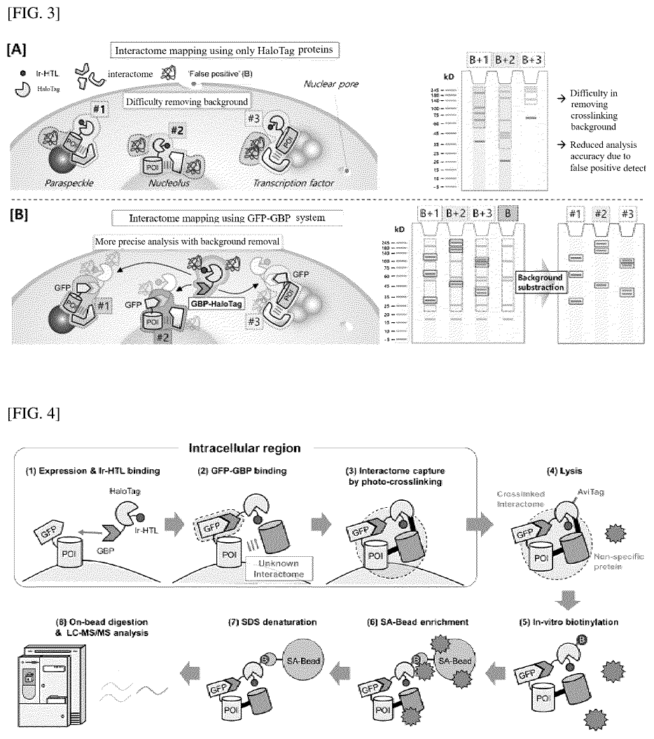

An example of the assay method of the present invention is illustrated in FIG. 4.

The present invention will be described in detail below through examples. These examples are merely for illustrating the present invention, and the scope of the present invention is not limited by these examples.

### [Experimental Example] Proof of Reliability of Interactome Analysis Method

In order to prove the reliability of the analysis method of the present invention, PTBP1, whose interactome is somewhat known, was selected as the protein of interest (POI).

Thereafter, analysis was performed in the following order.

(1) Two constructs, PTBP1-GFP and GBP-HaloTag-NLS, were expressed through transfection of cells using TurboFect (ThermoFisher Scientific) reagent (using 2 uL per 1000 ng of DNA).

TABLE 1

| Plasmid name | | Promotor/ |
|---|---|---|
| (expected size) | Features | vector |
| PTBP1-EGFP | NotI-PTBP1-BamHI-EGFP-STOP- | CMV/pCDNA3 |
| (86.6 kDa) | XhoI | |
| GBP-V5-Halotag- | KpnI-GBP-BamHI-V5-Halotag-TEV | CMV/pCDNA5 |
| AP-NLS | site-AP-NotI-NLS-STOP-XhoI | |
| (54.2 kDa) | | |

(2) incubating for 2 hours at 37° C. under 5% carbon dioxide conditions with media containing 2 uM of Ir-HTL, which is a photocatalyst, and then cultivating for an additional 8 hours with media without photocatalyst, targeting GBP-HaloTag-NLS and then removing unbound photocatalysts

(3) binding of GBP-HaloTag-NLS-Iridium to GFP-PTBP1

(4) photo-crosslinking chemical reaction under room temperature conditions between PTBP1 and the interactome due to the photoactivity of the iridium catalyst

(5) after reaction, cell lysis using RIPA lysis buffer at 4° C.

(6) in-vitro biotinylation of HaloTag protein to separate photo-crosslinked proteins.

(7) removing free biotin remaining in solution using AMICON filter.

(8) separating biotinylated HaloTag protein from lysate using streptavidin magnetic beads (Enrichment)

(9) removing non-specifically adsorbed proteins due to ionic and hydrophobic bonds by washing with 10% SDS solution (SDS denaturation)

(10) reacting the cross-linked protein attached to the beads with 10 mM Dithiothreitol (DTT) solution at 37° C. for 1 hour (reduction reaction)

(11) after removing the DTT solution, reacting with 55 mM Iodoacetamide (IAM) solution at 37° C. for 1 hour while blocking light (alkylation reaction)

(12) on-bead trypsin digestion of proteins using 20 μg/mL trypsin solution.

(13) after desalting the digested peptide solution with STAGE Tips, LC-MS/MS (analysis equipment: Thermo-Scientific Q Exactive Plus equipped with a nanoelectrospray ion source) analysis

* As the STAGE Tips, refer to: Rappsilber, J., Mann, M., Ishihama, Y. Protocol for micro-purification, enrichment, pre-fractionation and storage of peptides for proteomics using Stage Tips. Nat. Prot. 2, 1896-1906 (2007).

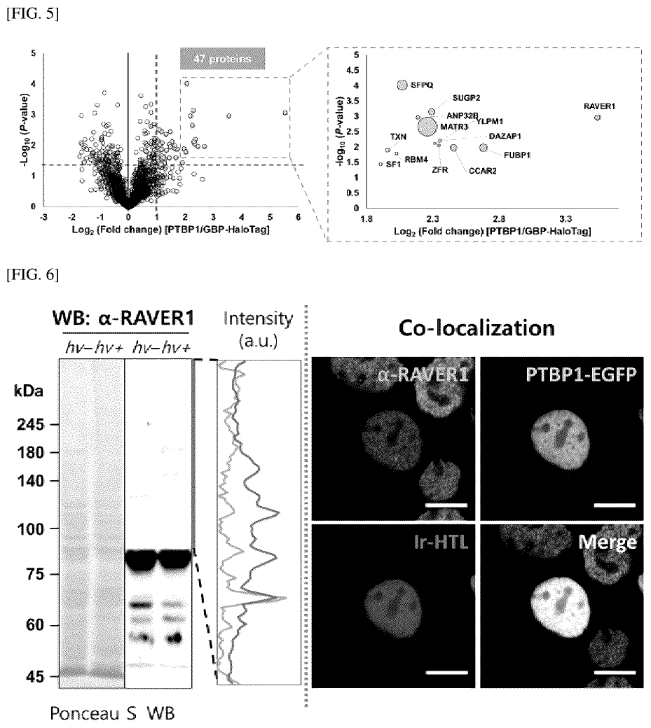

The analysis results of the proteins are visualized in a volcano plot, and the detected signal is more than twice that of the comparison group (log₂(fold change)>1), and as a result of selecting proteins with a reliability of more than 95% (−log₂(P-value)>1.3) in repeated experiments, RAVER1, MART3, and SFPQ, known as interactome proteins of PTBP1, were detected (FIG. 5).

In addition, when an experiment was conducted under photo-crosslinking conditions in PTBP1 using RAVER1 antibody to confirm the interaction of PTBP1-RAVER1, in case that PTBP1 was targeted by a photocatalyst (Ir-HTL), it was confirmed that photo-crosslinking actually occurred in RAVER1 as well. Also, it was confirmed that the positions on the microscope showed that the signals of PTBP1, RAVER1, and photocatalysts overlapped well (FIG. 6).

1. A kit for analyzing an interactome, comprising:
i) a first vector comprising a protein of interest (POI) coding portion and a fluorescent protein coding portion;
ii) a second vector comprising a fluorescent protein binding protein coding portion and a HaloTag coding portion; and
iii) a photocatalyst comprising a HaloTag targeting ligand (HTL).

2. The kit of claim 1, wherein the fluorescent protein includes a green fluorescent protein (GFP).

3. The kit of claim 1, wherein the fluorescent protein binding protein includes a green fluorescent protein binding protein (GBP).

4. The kit of claim 1, wherein the photocatalyst includes a conjugate of a metal and a Halotag ligand (HTL).

5. The kit of claim 4, wherein the metal includes iridium.

6. The kit of claim 1, wherein the kit is for analyzing the interactome within the cell.

7. The kit of claim 1, wherein the second vector further includes a nuclear localization sequence (NLS) coating portion.

8. A method for analyzing an interactome using a photo-crosslinking using the kit for analyzing the interactome of claim 1, the method comprising the steps of:
i) expressing in a cell a first construct comprising a protein of interest (POI) and a fluorescent protein, and a second construct comprising a fluorescent protein binding protein, a HaloTag, and a nuclear localization sequence, by using the first vector and the second vector;
ii) treating the cell with a photocatalyst comprising a HaloTag targeting ligand;
iii) irradiating a light source to induce a photo-crosslinking chemical reaction between the POI and the interactome; and
iv) extracting photo-crosslinked proteins.

9. The method of claim 7, wherein the method comprises, after the step iii), the step of lysing the cell.

10. The method of claim 7, wherein the method comprises, after the step iv), the step of analyzing a protein including the interactome through mass spectrometry.
